SEQUENCE LISTING (17 sequences)

SEQ ID NO: 1 - RNA, 19 nt, Artificial Sequence
  Synthetic Oligonucleotide
cgugcaaagugguauccua

SEQ ID NO: 2 - RNA, 19 nt, Artificial Sequence
  Synthetic Oligonucleotide
uaggauaccacuuugcacg

SEQ ID NO: 3 - RNA, 19 nt, Artificial Sequence
  Synthetic Oligonucleotide
cgugcaaagugguauccua

SEQ ID NO: 4 - RNA, 19 nt, Artificial Sequence
  Synthetic Oligonucleotide
uaggauaccacuuugcacg

SEQ ID NO: 5 - RNA, 19 nt, Artificial Sequence
  Synthetic Oligonucleotide
cgugcaaagugguauccua

SEQ ID NO: 6 - RNA, 19 nt, Artificial Sequence
  Synthetic Oligonucleotide
uaggauaccacuuugcacg

SEQ ID NO: 7 - RNA, 19 nt, Artificial Sequence
  Synthetic Oligonucleotide
cgugcaaagugguauccua

SEQ ID NO: 8 - RNA, 19 nt, Artificial Sequence
  Synthetic Oligonucleotide
uaggauaccacuuugcacg

SEQ ID NO: 9 - RNA, 19 nt, Artificial Sequence
  Synthetic Oligonucleotide
cgugcaaagugguauccua

SEQ ID NO: 10 - RNA, 19 nt, Artificial Sequence
  Synthetic Oligonucleotide
uaggauaccacuuugcacg

SEQ ID NO: 11 - RNA, 38 nt, Artificial Sequence
  Synthetic Oligonucleotide
mcrgmurgmcrararargmurgrgmurarumcrcmura

SEQ ID NO: 12 - RNA, 19 nt, Artificial Sequence
  Synthetic Oligonucleotide
uaggauaccacuuugcacg

SEQ ID NO: 13 - RNA, 19 nt, Artificial Sequence
  Synthetic Oligonucleotide
cgugcaaagugguauccua

SEQ ID NO: 14 - RNA, 19 nt, Artificial Sequence
  Synthetic Oligonucleotide
uaggauaccacuuugcacg

SEQ ID NO: 15 - RNA, 19 nt, Artificial Sequence
  Synthetic Oligonucleotide
cgugcaaagugguauccua

SEQ ID NO: 16 - RNA, 20 nt, Artificial Sequence
  Synthetic Oligonucleotide
ccgugcaaagugguauccua

SEQ ID NO: 17 - RNA, 19 nt, Artificial Sequence
  Synthetic Oligonucleotide
cgugcaaagugguauccua

(19) United States
(12) Patent Application Publication
(10) Pub. No.: US 2016/0152979 A1
(43) Pub. Date: Jun. 2, 2016

(54) NOVEL COMPOUNDS

(71) Applicants: QBI ENTERPRISES LTD., Nes Ziona, IL (US); BIO-LAB LTD., Jerusalem (IL)

(72) Inventors: Sharon AVKIN-NACHUM, Nes Zionna (IL); Abram BECKER, Paris (FR); Tirtsa KLEINMAN, Jerusalem (IL); Myriam LEFOIX, Le Bardon (FR); Jean-Christophe TRUFFERT, Saint-Prest (FR)

(73) Assignees: QBI ENTERPRISES LTD., Nes Ziona (IL); BIO-LAB LTD., Jerusalem (IL)

(21) Appl. No.: 15/014,084

(22) Filed: Feb. 3, 2016

Related U.S. Application Data
(63) Continuation-in-part of application No. 14/417,796, filed on Jan. 28, 2015.
(60) Provisional application No. 61/699,882, filed on Sep. 12, 2012.
(60) Provisional application No. 62/111,692, filed on Feb. 4, 2015.

(51) Int. Cl. C12N 15/113; A61K 31/122; A61K 47/48

(52) U.S. Cl. CPC C12N 15/113; A61K 47/48023; A61K 31/122; A61K 47/48215; C12N 2310/14; C12N 2310/351

(57) ABSTRACT

Disclosed herein are novel compounds having the general formula (I):

Q-L-A1 (I)

wherein Q is a Q10 moiety, such as ubiquinone; L, which is optionally included, is a linker selected from the group consisting of polyesters, polyethers, polyamines, polyamides, peptides, carbohydrates, lipids, C₃₋₁₂ alkyl straight chain based linkers, polyethylene glycols and other polymeric compounds; and A1 is a nucleotide moiety. Disclosed are also pharmaceutical compositions comprising such compounds or pharmaceutically acceptable salts thereof. Further disclosed is the use of such compounds in the treatment of cancer and/or a cancer related medical condition.

### CROSS-REFERENCE TO RELATED APPLICATIONS

This is a Continuation-in-Part of co-pending U.S. patent application Ser. No. 14/417,796, filed Jan. 28, 2015, which is the U.S. National Stage of International Patent Application No. PCT/US2013/059345, filed Sep. 12, 2013, and which claims the benefit of U.S. Patent Application No. 61/699,882, filed Sep. 12, 2012. This application additionally claims the benefit of U.S. Patent Application No. 62/111,692, filed Feb. 4, 2015. The contents of the foregoing patent applications are incorporated by reference herein in their entirety.

### TECHNICAL FIELD

Provided herein are novel compounds and pharmaceutical compositions comprising the same. Provided herein are also the novel compounds for use in the treatment of various medical conditions, use of the novel compounds for the manufacture of a medicament for treatment of various medical conditions and methods for treatment of said conditions, wherein the novel compounds are administered. The novel compounds are conjugates comprising a nucleotide moiety, such as an oligonucleotide moiety.

### BACKGROUND

Oligonucleotides are valuable tools in the modulation of gene expression in a sequence specific manner. The expression and function of a variety of proteins have been successfully modified using an assortment of oligonucleotide-based approaches. Some molecules modulate protein expression (e.g. those acting via RNA interference (RNAi), antisense (AS), ribozymes, activating RNA (RNAa) and the like), and others modulate protein function (e.g. aptamers). Oligonucleotides represent a rapidly developing class of therapeutically active agents.

However, many times, insufficient properties, such as pharmacokinetic properties and cellular uptake, for oligonucleotides alone have prevented successful therapeutic use of oligonucleotides. Oligonucleotide conjugates, wherein oligonucleotides are attached to different ligands, have therefore been studied.

U.S. Pat. Nos. 6,172,208; 6,825,338; 8,426,377; 6,919,439; 7,833,992 and 8,252,755 disclose oligonucleotides modified with conjugate groups. Conjugate moieties include cell penetrating moieties and cell targeting moieties e.g. ligands, vitamins, cholesterol and peptides. The conjugate moiety may be covalently attached to a nucleic acid molecule, such as a siNA molecule, directly or via an e.g. alkyl or peptidic linker. The linker itself may be stable or biodegradable.

Further oligonucleotide conjugates are discussed in a review article by J. Winkler, Ther. Deliv. (2013) 4(7).

Provided herein are compounds that enable efficacious delivery of therapeutically active polynucleotides or oligonucleotides.

### BRIEF SUMMARY

It has now been realized by the present inventors that compounds according to the following general formula (I) meet inter alia the aforementioned objective.

Disclosed herein are compounds having the general formula (I):

Q-L-A1 (I)

wherein:

Q is a Q10 moiety;

L, which is optionally included, is a linker selected from the group consisting of polyesters, polyethers, polyamines, polyamides, peptides, carbohydrates, lipids, C₃₋₁₂ alkyl straight chain based linkers, polyethylene glycols and other polymeric compounds; and

A1 is a nucleotide moiety;

and pharmaceutically acceptable salts thereof.

Disclosed herein are also the above compounds for use as a pharmaceutical.

Further disclosed are pharmaceutical compositions comprising such said compound as active ingredient in association with a pharmaceutically acceptable adjuvant, diluent or carrier.

Also disclosed are such compounds for use in the treatment of cancer and/or a cancer related medical condition.

Disclosed is also the use of such compounds for the manufacture of a medicament for treatment of cancer and/or a cancer related medical condition.

Disclosed are further methods for treatment of cancer and/or a cancer related medical condition, comprising administering to a patient in need of said treatment a therapeutically effective amount of compound (I).

### BRIEF DESCRIPTION OF THE DRAWINGS

FIG. 1 illustrates examples of a few different linkers.

FIG. 2 illustrates dose-dependent knockdown of Renilla Luciferase activity for Q10-conjugated siRNA.

FIG. 3 shows gel migration patterns confirming stability of the Q10-conjugated siRNA.

FIG. 4 illustrates the result of comparison of pharmacokinetics, and demonstrates that the residual level of the Q10-conjugated siRNA was at least 25 fold higher compared to the non-conjugated siRNA and about 3-10 fold higher compared to the Sphingolipid conjugated siRNA, respectively.

FIG. 5 shows the results of fluorescence analysis demonstrating that Q10-conjugated siRNA penetrated into the cells and remained up to 72 hours while the non-conjugated siRNA was not detected at that time point, and also that Q10-siRNA is not co-localized with tested organelles.

FIG. 6 illustrates that a shift in cell signal can be observed in cells treated with the conjugated siRNA already after 2 h (middle panels) suggesting binding of the Q10-conjugated siRNA to the cells and that this shift is increased reaching full staining of most of the cells after 6 h (bottom panels). This shift is hardly observed in the histogram for the cells that were treated with the non-conjugated siRNA.

The drawings form part of the present specification and are included to further demonstrate certain aspects of the present disclosure. They are not in any way intended to limit the scope of the invention as claimed.

### BRIEF DESCRIPTION OF THE DESCRIBED SEQUENCES

The nucleic acid sequences provided herewith are shown using standard letter abbreviations for nucleotide bases as defined in 37 C.F.R. 1.822. Only one strand of each nucleic acid sequence is shown, but the complementary strand is understood as included by any reference to the displayed strand. The Sequence Listing is submitted as an ASCII text file named 3065_29_3 seq_list.txt, created Feb. 3, 2016, about 14.1 KB, which is incorporated by reference herein.

### DETAILED DESCRIPTION

It is readily apparent to one skilled in the art that various embodiments and modifications may be made to the disclosures herein without departing from the scope of the claims.

All references mentioned herein are indicative of the level of knowledge of those skilled in the art. All references mentioned herein are incorporated by reference to the same extent as if each individual reference had been specifically and individually indicated to be incorporated by reference. All references mentioned herein are to be regarded as an integral part of the present writ.

Herein, the term “alkyl” includes saturated aliphatic groups, including straight-chain alkyl groups (typically C₁₋₁₂), branched-chain alkyl groups (typically C₃₋₁₂), cycloalkyl groups (typically C₃₋₈), alkyl substituted cycloalkyl groups, alkylaryl groups and cycloalkyl substituted alkyl groups, unless otherwise specified.

The terms “alkenyl” and “alkynyl” include unsaturated aliphatic groups, having at least one double and triple bond respectively, of C₂₋₁₀ length and a possible substitution pattern analogous to the said alkyls, unless otherwise specified.

The term “alkoxy” includes alkyl, as defined above, covalently linked to an oxygen atom, unless otherwise specified. Methoxy is the most preferred alkoxy group.

As mentioned above, the present disclosure relates to compounds having the following general formula (I).

Q-L-A1 (I)

The compounds of general formula (I) are conjugates. The term “conjugate” as used herein relates to a chemical compound, i.e. the compound of formula (I) that has been formed by the joining of two compounds, namely the two moieties Q and A1. Optionally, a linker may be used in order to join Q to A1 via covalent attachment.

In the general formula (I), “Q” denotes a Q10 conjugate moiety, or in other words a part of the conjugate consisting of Q10.

In the context of the present disclosure, Q10 denotes the coenzyme Q10 (which may also be denoted Q₁₀, CoQ, CoQ10 or CoQ₁₀). Q10 may exist in three different states: 1) oxidized form, ubiquinone; 2) semiquinone or ubisemiquinone; and 3) reduced form, ubiquinol. The compounds of formula (I) may comprise any of these different states of Q10, or modifications thereof.

In some embodiments, the following compound, which is ubidecarenone (CAS RN 303-98-0), is used:

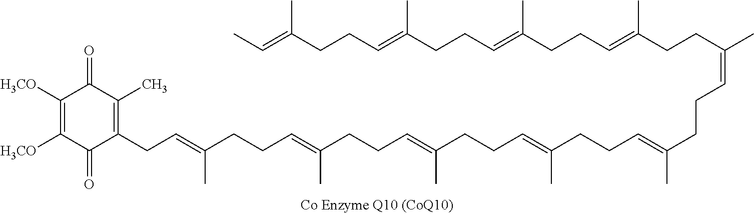

In formula (I), L denotes a linker. As mentioned above, the linker is optional, i.e. it may be either present or absent, which means that in some embodiments, wherein the linker is not present, the compounds will have the simplified general formula (I′):

Q-A1 (I′)

The linker, L, if present, is defined supra.

Herein C₃₋₁₂ denotes a hydrocarbon having from three to twelve carbon atoms, including three, twelve and any integer there between, and this nomenclature, as well as the nomenclature 3-12, having the same meaning, is used analogously herein. For example, isopropyl, 2-n-butyl, 2-n-pentyl groups etc. are encompassed by the expression C₃₋₁₂ alkyl straight chain, as said expression is not related to the binding site of the straight chain in question.

In some embodiments, the linker “L” is selected from the group consisting of polylactate, triethyloxy-glycol phosphoramidite, ethanediol-phosphoramidite, hexanediol phosphoramidite, nonanediol phosphoramidite, propane-diol phosphoramidite, hexa-ethyloxy-glycol-phosphoramidite and abasic carbohydrate phosphoramidite.

In some embodiments, the linker “L” is selected from the group consisting of polyethylene glycols, including modified polyethylene glycols.

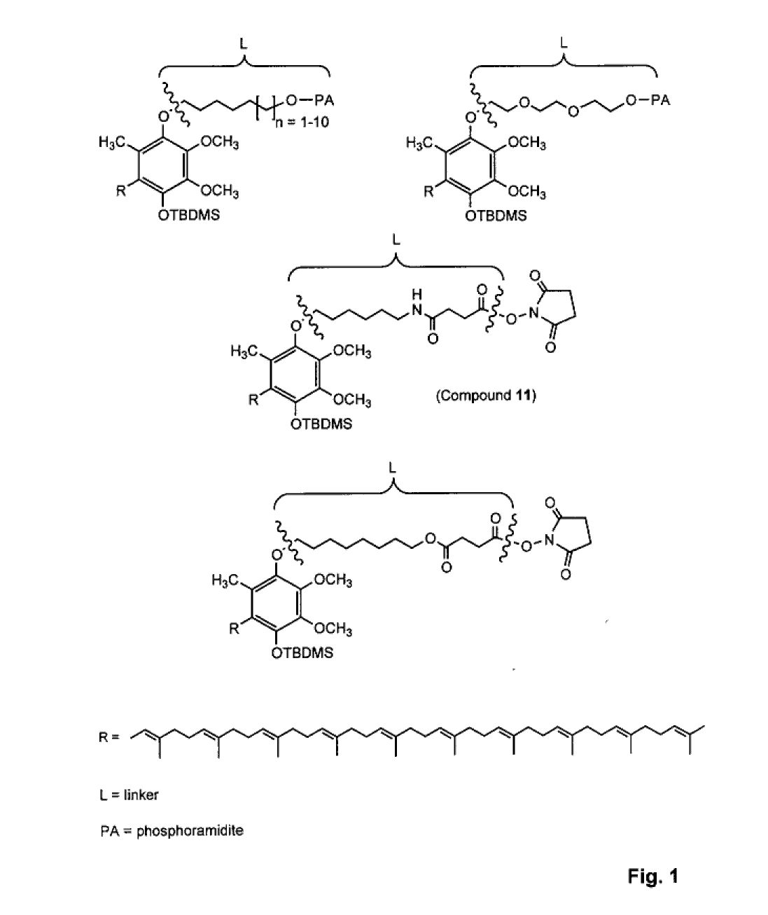

Examples of different linkers are illustrated in FIG. 1, which includes compounds wherein the linker is N-hydroxysuccinimide (NHS) ester, wherein the linker is another ester, and wherein the linker is an amidite.

In formula (I), A1 is a nucleotide moiety, or nucleic acid moiety. In this context, the terms “nucleotide moiety”, “nucleic acid moiety”, “nucleic acid compound moiety” and “nucleic acid molecule moiety” may be used interchangeably. This moiety may be an oligomer (oligonucleotide) or polymer (polynucleotide) comprised of unmodified ribonucleic acid (RNA) or deoxyribonucleic acid (DNA) and/or modified RNA, and/or modified DNA, and/or nucleotide analogues.

The term oligonucleotide herein refers to a nucleic acid molecule containing up to 100 nucleotide bases, and the term polynucleotide refers to a nucleic acid molecule containing at least 100 nucleotide bases.

A1 is preferably an oligonucleotide moiety.

Exemplary oligonucleotide moieties are selected from the group consisting of siRNA, ASO, miRNA, antimir, ribozyme, mRNA and aptamers.

siRNA herein denotes small interfering RNA, also called short interfering RNA or silencing RNA, as further discussed below.

ASO, allele-specific oligonucleotide, is a short piece, typically of 15-21 nucleotide bases in length of synthetic DNA complementary to the sequence of a variable target DNA, i.e. the allele for which it is specific.

miRNA, or microRNA, and antimir, anti-miRNA oligonucleotides (AMOs), are also discussed below.

Ribozymes are ribonucleic acid enzymes, also called catalytic RNA.

mRNA denotes messenger RNA.

Aptamers are oligonucleotide or peptide molecules that bind to a specific target molecule. Oligonuleotide aptamers, in particular RNA aptamers, are especially preferred herein.

The oligonucleotides or polynucleotides used may be chemically or biologically synthesized, using techniques known to persons skilled in the art.

Herein, the terms “mRNA polynucleotide sequence”, “mRNA oligonucleotide sequence”, “mRNA sequence” and “mRNA” are used interchangeably; and the same applies to the other different oligonucleotides mentioned here above.

In some embodiments, the A1 moiety is an ssNA molecule, i.e. a single stranded nucleic acid molecule. An example thereof is ssRNA.

In some embodiments, the A1 moiety is a dsNA molecule, i.e. a double stranded nucleic acid molecule.

The terms “dsNA” and “ssNA” also include saNA (short activating nucleic acid) molecules, which induce target gene expression at the transcriptional and/or post-transcriptional level. Activating NAs may induce potent transcriptional activation of associated genes by targeting gene promoters.

More precisely, the term “dsNA” refers to double stranded nucleic acid and relates to a molecule with two strands of anti-parallel oligonucleotides forming a duplex, in part or in full, by base pairing. Each oligonucleotide may include RNA, DNA and/or modified nucleotides and/or nucleotide analogues. The two strands can be of identical lengths (symmetric dsNA) or of different lengths (asymmetric dsNA). In some embodiments, the duplex includes an antisense strand that, counting from its 5′ end, has a position 1, 2 and/or 3 mismatch to the target RNA. The sense strand may be fully matched or partially matched to the antisense strand. In some embodiments at least a portion of the sequence of the antisense strand is complementary to a consecutive sequence in the target mRNA.

In a dsRNA molecule, at least one strand of the duplex or double-stranded region is substantially homologous to or substantially complementary to a target RNA molecule. The strand complementary to a target RNA molecule (e.g. mRNA) is the “antisense, or guide, strand;” the strand homologous to the target RNA molecule is the “sense, or passenger, strand,” and is also complementary to the dsNA antisense strand. dsNAs may also contain additional sequences; non-limiting examples of such sequences include linking sequences, or loops, as well as additional stem structures and other folded structures (i.e. aptamers).

A dsRNA compound useful for therapy is a duplex oligoribonucleotide in which the antisense strand is substantially complementary to a 15-49 consecutive nucleotide segment of the mRNA polynucleotide sequence of a target gene, and the sense strand is substantially complementary to the antisense strand. In general, some deviation from the target mRNA sequence is tolerated without compromising the dsRNA activity. A dsRNA as disclosed herein inhibits gene expression on a post-transcriptional level usually by destroying the mRNA. Without being bound by theory, siRNA may target the mRNA for specific cleavage and degradation and/or may inhibit translation from the targeted message.

The dsNA is typically blunt ended at one or both termini, i.e. has no overhangs. The dsNA may optionally include a nucleotide (non-modified and/or modified) or a non-nucleotide overhang (e.g. carbon chains) at one or more terminus, e.g. the 3′ termini. —CH₂CH₂CH₂Pi (‘C3Pi’; Pi=phosphate) and C3Pi-C3Pi overhangs, cf. US 2013/0035368, are examples thereof.

In some embodiments, one or more nucleotide or non-nucleotide moieties are present at the 5′ terminus of the sense strand and/or antisense strand. Such a 5′ terminal moiety is also known as a “cap”.

The term dsNA includes “siNA”, which are double stranded molecules comprising unmodified and modified nucleotides and/or nucleotide analogues. “siNA”, which are small interfering dsNA (generally 15-40 bp) can include siRNA which are small interfering dsRNA molecules. Typically, siNA (including siRNA) are chemically synthesized as 8-40 mers, preferably comprising a central 15-21 bp duplex region. The dsNA molecules are preferably capable of inducing modulation of target gene expression via the RNA interference (RNAi) pathway.

The complementary strands of a dsNA and siRNA herein may be linked to form a hairpin, or stem-loop, structure, such as a short hairpin NA (shNA).

Several duplexes can be linked together, each duplex targeting different regions of the same or different mRNA (cf. U.S. Pat. No. 8,362,229).

The term dsNA also includes the terms “microRNA” or “miRNA” or “miR” that refer to a class of naturally occurring gene regulatory small RNAs, typically 21-23 nucleotides in length. miRNAs have been implicated in a wide range of functions and e.g. some cancers are associated with up- or down-regulation of certain miRNAs. The term “microRNA” includes mature miRNAs, pre-miRNAs and pro-miRNAs and variants thereof, which may be naturally occurring or synthetic (miRNA mimetics) that can be modified analogously to any synthetic NA compound described herein. The term “anti-miR” or “antagomir” refers to a NA molecule that can block miRNA activity.

When the A1 moiety is a double stranded oligonucleotide, Q may be covalently attached, optionally via the linker L, at the 3′-end of either the sense strand, the antisense strand, or both strands of a nucleic acid molecule “A1”. Analogously, the Q moiety may alternatively be attached at one of or both the 5′-ends of the said strands. Furthermore, the Q moiety may alternatively be attached at both the 3′-end and 5′-end of the sense strand, the antisense strand, or both strands of A1. The Q moiety may also be attached to any combination of the 3′-end the sense strand of A1, the 3′-end of the antisense strand of A1, the 5′-end of the sense strand of A1, and/or the 5′-end of the antisense strand of A1.

In some embodiments A1 is a siRNA molecule, and in some of these it is a modified siRNA molecule. In other embodiments, at least one of the ribonucleotides in the siRNA is substituted with a modified nucleotide, a nucleotide analogue or an unconventional moiety.

The preferred dsNA molecule for the A1 moiety of the compounds of formula (I) has the following structure:

5′ Z″-N1-(N)ₓ-Z 3′ (antisense strand)
3′ Z′-N2-(N′)_{y}-z″ 5′ (sense strand)

wherein each of N1, N2, N and N′ independently is an unmodified nucleotide, a modified nucleotide, nucleotide analogue or an unconventional moiety;

wherein each of (N)ₓ and (N′)_{y} is an oligonucleotide in which each consecutive N and N′ is joined to the adjacent N or N′ by a covalent bond;

wherein each of x and y is independently an integer between 14 and 48;

wherein at least a portion of the sequence of N2-(N′)_{y} is complementary to at least a portion of the sequence of N1-(N)ₓ and at least a portion of the sequence of (N)ₓ is complementary to a consecutive sequence in a target RNA;

wherein N2 is covalently bound to (N′)_{y};

wherein N1 is covalently bound to (N)ₓ and is matched or mismatched to the target mRNA;

wherein z″ is a covalently attached optionally present capping moiety or a covalent bond to the Q conjugate moiety or to the linker L;

wherein each of Z, Z′ and Z″ is independently optionally present as covalently attached 1-5 consecutive nucleotides, 1-5 consecutive nucleotide analogues or 1-5 consecutive non-nucleotide moieties, or a covalent bond to the Q conjugate moiety or to the linker L, or a combination thereof.

In some embodiments, the sequence of (N′)_{y} is fully complementary to the sequence of (N)ₓ.

In some embodiments, the sequence of (N)ₓ is fully complementary to a consecutive sequence in the target RNA.

In some embodiments the dsNA hence comprises a DNA moiety or a mismatch to the target at position 1 of the antisense strand (5′ terminus).

Where N1 is mismatched to the target mRNA it is a moiety selected from the group consisting of natural uridine, a modified uridine, deoxyribouridine, ribothymidine, deoxyribothymidine, natural adenosine, modified adenosine, deoxyadenosine, adenosine pyrazolotriazine nucleic acid analogue and deoxyadenosine pyrazolotriazine nucleic acid analogue.

In preferred embodiments, each nucleotide is independently an unmodified or a modified ribonucleotide (e.g. 2′O-alkyl, 2′deoxy) or a nucleotide analogue (e.g. L-DNA, L-RNA, TNA, 2'S′ linked, UNA and the like).

In some embodiments, each of Z, Z′ and Z″ comprises 1-2 consecutive non-nucleotide moieties. Here the sense strand, the antisense strand, or both, independently include 1-5 nucleotide or non-nucleotide moieties at the 3′ terminus. The sense strand preferably includes a dinucleotide overhang, C3OH or C3Pi moiety (Z′). The antisense strand preferably includes a dinucleotide overhang, C3Pi-C3OH or C3Pi-C3Pi moiety (Z and/or Z″).

Preferably the covalent bond joining each consecutive N or N′ is independently a phosphodiester bond, a phosphorothioate bond or a modified internucleotide linkage.

In some embodiments, x and y are of the same lengths, i.e. x=y, and both x and y is then an integer from 14 to 48, limits included, or from 17 to 40, limits included, or from 18 to 25, limits included. In some embodiments both x and y are 18.

In some embodiments, x and y are of different lengths. In some of these embodiments, x is an integer from 18 to 25, limits included, and y is an integer from 15 to 17, limits included.

In some embodiments, the sequence of (N′)_{y} is fully complementary to the sequence of (N)ₓ, and the sequence of (N)ₓ is fully complementary to the target RNA. The sequence of (N′)_{y} may also be fully complementary to the sequence of (N)ₓ and the sequence of (N)ₓ partially complementary to the target RNA. In such compounds, for example, the 5′ terminal nucleotide of the antisense strand (N)ₓ is mismatched to the target RNA. The nucleotide on the sense strand opposite the 5′ terminal nucleotide of the antisense strand may be complementary or not.

In certain embodiments, at least one modified ribonucleotide comprises a 2′ sugar modification, selected from the group consisting of 2′O-alkyl sugar modification, for example a 2′O-methyl (2′OMe) sugar modification, 2′deoxyfluoro (2′ fluoro or 2′F) sugar modification, 2′-O-methoxyethyl (2′MOE) sugar modification and a 2′-amino sugar modification. In some embodiments, one or more pyrimidines is 2′O-methyl sugar modified or 2′ deoxyfluoro sugar modified.

In some embodiments, one or more up to about 12 of N and N′ in the modified molecule is a nucleotide analogue.

In some embodiments, at least one nucleotide analogue is present in the sense strand, (N′)_{y}, the nucleotide analogue selected from a 2′5′ linked nucleotide (i.e. 2′5′ linked RNA or DNA), a threose nucleic acid (TNA), a pyrazolotriazine nucleotide or a mirror nucleotide (i.e. L-DNA or L-RNA). In some embodiments, x=y=18 and a 2′5′ linked nucleotide is present in positions (5′>3′) 15, 16, 17, 18 and/or 19. In some such embodiments the compound comprises a 2′5′ linked nucleotide, TNA or a mirror nucleotide in at least one of positions 6, 7 or 8 in the antisense strand, (N)ₓ. In some embodiments, a pyrazolotriazine nucleotide analogue is present in the antisense strand in at least one of positions 4 to 7 (5′>3′). In some embodiments, an unlocked nucleic acid (UNA) is present in the antisense strand in at least one of positions 4 to 7 (5′>3′).

As mentioned above, the nucleotides forming the A1 moiety may be either unmodified or modified, independently of each other, i.e. one, two, more, or all of the nucleotides in an A1 moiety may be either unmodified or modified. Modifications are also applicable for nucleotide analogues.

An “unmodified nucleotide” is one naturally observed in cellular nucleic acids and composed of a nitrogenic base (A, G, T, C, U), a D-deoxyribose or D-ribose sugar moiety (furanose) and at least one phosphate group, which may make up an internucleoside linkage (backbone).

A “modified nucleotide” comprises naturally occurring (in the context of nucleic acids) D-ribose or D-deoxyribose and a nitrogenic base (A, G, T, C, U) components that are independently chemically modified, i.e. a naturally occurring nitrogenic base may be combined with a chemically modified sugar or internucleoside linkage, vice versa or both can be chemically modified. Note that in the context of siRNA, miRNA or mRNA, DNA nucleotides and uridine in the context of a deoxyribose (2′H) are considered as modified nucleotides. Likewise, a naturally occurring but not abundant nucleotide variant, such as pseudo-U, inosine (I), or a nucleotide with 2′ methoxy (2′O-Me) or a 2′ fluoro (2′F) modified sugar, is also considered as a modified nucleotide.

A “nucleotide analogue” independently comprises a base and/or sugar substitution. Nucleotide analogues may contain further modifications either in the base, internucleotide and/or the sugar component. Non-limiting examples of nucleotide analogues include a peptide nucleic acid (PNA), in which the sugar-backbone of a nucleotide is replaced with an amide containing backbone, in particular an aminoethylglycine backbone; a morpholino nucleic acid, in which the furanose ring is replaced with a morpholine ring; a cyclohexenyl nucleic acid (CeNA) where the furanose ring is replaced with a cyclohexenyl ring; a nucleic acid comprising bicyclic sugar moiety (BNAs), such as “Locked Nucleic Acids” (LNAs) in which the 2′-hydroxyl group of the ribosyl sugar ring is linked to the 4′ carbon atom of the sugar ring thereby forming a 2′-C,4′-C-oxymethylene linkage to form the bicyclic sugar moiety, a 2′-0,4′-ethylene-bridged nucleic acid (ENA) and the like; a threose nucleic acid (TNA) in which the hydroxylpentofuranosyl sugar moiety is replaced with threose sugar moiety; an arabino nucleic acid (ANA) in which the ribose sugar moiety is replaced with arabinose sugar moiety; an unlocked nucleic acid (UNA), in which the ribose ring is replaced with an acyclic analogue, lacking the C2′-C3′ bond; a mirror nucleotide in which the typical D-ribose (or deoxyribose) ring is replaced with a L-ribose (or deoxyribose) ring, thus forming a nucleotide which is a mirror image (having opposite chirality).

The nucleotide analogues described may be further modified as described above for “modified nucleotide”.

In some embodiments, the oligonucleotides disclosed herein include nucleotide analogues with 2′-sugar substituent groups that may be incorporated in the arabino (up) position or ribo (down) position. An example of 2′-arabino modification is 2′-F (2′-F-arabino modified nucleotide is typically referred to as fluoroarabino nucleic acid (FANA)).

Modified internucleoside linkages (backbone): The nucleoside subunits of the nucleic acids disclosed herein may be linked to each other by phosphodiester bonds. The phosphodiester bond may be optionally substituted with other linkages. For example, phosphorothioate, thiophosphate-D-ribose entities, triester, thioate, 2′-5′ bridged backbone, PACE, 3′-(or -5′)deoxy-3′-(or -5′)thio-phosphorothioate, phosphorodithioate, phosphoroselenates, 3′-(or -5′)deoxy phosphinates, borano phosphates, 3′-(or -5′)deoxy-3′-(or 5′-)amino phosphoramidates, hydrogen phosphonates, phosphonates, borano phosphate esters, phosphoramidates, alkyl or aryl phosphonates and phospho-triester modifications such as alkylphosphotriesters, phosphotriester phosphorus linkages, 5′-ethoxyphosphodiester, P-alkyloxyphosphotriester, methylphosphonate, and nonphosphorus containing linkages for example, carbonate, carbamate, silyl, sulfur, sulfonate, sulfonamide, formacetal, thioformacetyl, oxime, methyleneimino, methylenemethylimino, methylenehydrazo, methylenedimethylhydrazo and methyleneoxymethylimino linkages.

Additional internucleoside modifications include those having morpholino linkages (formed in part from the sugar portion of a nucleoside); siloxane linkages; sulfide, sulfoxide and sulfone linkages; formacetyl and thioformacetyl linkages; methylene formacetyl and thioformacetyl linkages; riboacetyl linkages; alkene containing linkages; sulfamate linkages; methyleneimino and methylenehydrazino linkages; amide linkages; and other linkages having mixed N, O, S and CH₂ component parts.

Exemplary heteroatom internucleoside linkages are —CH₂—NH—O—CH₂—, —CH₂—N(CH₃)—O—CH₂— (methylene(methylimino)), —CH₂—O—N(CH₃)—CH₂—, —CH₂—N(CH₃)—N(CH₃)—CH₂— and —O—N(CH₃)—CH₂—CH₂—.

Sugar modifications and sugar moieties in nucleic acid compounds disclosed herein may include 2′-hydroxylpentofuranosyl sugar moiety without any modification (2′OH). Alternatively, nucleic acid compounds of the disclosure may contain one or more substituted or otherwise modified sugar moieties (modified nucleotide). A preferred position for a sugar substituent group is the 2′-position not usually used in the native 5′ to 3′-internucleoside linkage. Other preferred positions are the 3′ and the 5′-termini. Preferred sugar substituent groups include: —OH; —F; —O—, —S— and —N-alkyl; —O—, —S— and —N-alkenyl and -alkynyl; and —O-alkyl-O-alkyl, wherein the alkyl, alkenyl and alkynyl may be substituted or unsubstituted C₁₋₁₀ alkyl or C₂₋₁₀ alkenyl and alkynyl, C₁₋₁₀ lower (C₁₋₃) alkyl, substituted lower alkyl, alkenyl, alkynyl, alkaryl, aralkyl (e.g. -propargyl, -propyl, -ethynyl, -ethenyl and propenyl). Other sugar modifications include methoxy (—OCH₃), methylthio (—SCH₃), —SCN, —OCN, —OCF₃, —SCF₃, aminopropoxy (—OCH₂CH₂CH₂NH₂), —O-allyl (—O—CH₂—CH═CH₂), —O[(CH₂)ₙO]ₘCH₃, —(CH₂)ₙOCH₃, —O(CH₂)ₙNH₂, —O(CH₂)ₙCH₃, —O(CH₂)ₙONH₂ and —O(CH₂)ₙON[(CH₂)ₙCH₃]₂, where n and m independently are from 1 to 10; 2′-methoxyethoxy, 2′-dimethylaminooxyethoxy (2′-O(CH₂)₂ON(CH₃)₂), —N-methylacetamide (2′-O—CH₂—C(═O)—N(H)CH₃), —Cl, —SOCH₃, —SO₂CH₃, —NO₂, —NH₂, imidazole, carboxylate, thioate, heterocycloalkyl, heterocycloalkaryl, aminoalkylamino, polyalkylamino and substituted silyl. Preferably the modified nucleotide comprises at least one 2′-OCH₃ sugar moiety. Modifications are also possible e.g. at the 3′ position of the sugar of a 3′ terminal nucleotide and the 5′ position of a 5′ terminal nucleotide.

Nucleobase modifications of the nucleic acid compounds disclosed herein may comprise “unmodified” or “natural” nucleobases including the purine bases adenine (A) and guanine (G), and the pyrimidine bases thymine (T), cytosine (C) and uracil (U). Nucleic acid compounds herein may optionally and independently contain one or more substituted or otherwise modified nucleobases, e.g. including 5-methyl- and 5-hydroxymethyl cytosine; xanthine and hypoxanthine (inosine); 2-aminoadenine; 6-methyl, 7-methyl and 2-propyl adenine and guanine and other C₁₋₁₀ alkyl derivatives thereof; 2-thiouracil, -thymine and -cytosine; 5-halo and 5-propynyl uracil and cytosine; 6-azo uracil, cytosine and thymine; 5-uracil (pseudouracil); 4-thiouracil; 8-halo, 8-NH₂, 8-SH, 8-S—C₁₋₁₀ alkyl and 8-OH adenine and guanine and other 8-substitutions thereof; 5-trifluoromethyl uracil and guanine and other 5-substitutions thereof; 2-F-adenine. Modified nucleobases may also include purine or pyrimidine base independently replaced with other heterocycles, e.g. 8-aza-, 7-deaza- and 3-deazaguanine and -adenine. Further examples include non-purinyl and non-pyrimidinyl nucleobases, e.g. 2-aminopyridine, 2-pyridone, triazine and pyrazolo bases (cf. WO 2013/179289).

The nucleic acid compounds disclosed herein may further comprise at least one unconventional moiety. The term “unconventional moiety” as used herein refers to an “abasic nucleotide” or an “abasic nucleotide analogue”. Such abasic nucleotide encompasses sugar moieties lacking a base or having other chemical groups in place of base at the 1′ position. The abasic nucleotide may comprise an abasic ribose, deoxyribose or dideoxyribose moiety, optionally modified as above, or an unnatural sugar (morpholino, altritol etc). Additionally, the abasic nucleotide may be a reverse abasic nucleotide, e.g. where a reverse abasic phosphoramidite is coupled via a 5′ amidite (instead of 3′ amidite) resulting in a 5′-5′ phosphate linkage.

Modifications can be made at terminal phosphate groups. Stabilization techniques, e.g. to stabilize the 3′-end of nucleic acid sequences, include [3-3′]-inverted deoxyribose; deoxyribonucleotide; [5′-3]-3′-deoxyribonucleotide; [5′-3′]-ribonucleotide; [5′-3]-3′-O-methyl ribonucleotide; 3′-glyceryl; [3′-5′]-3′-deoxyribonucleotide; [3′-3]-deoxyribonucleotide; [5′-2]-deoxyribonucleotide; [5′-5]-1,2-dideoxy-D-ribofuranose and [5-3′]-dideoxyribonucleotide. In addition such techniques can be combined with different internucleotide linkage modifications, sugar modifications and/or nucleobase modifications as described above. Exemplary chemically modified terminal phosphate groups include those shown below:

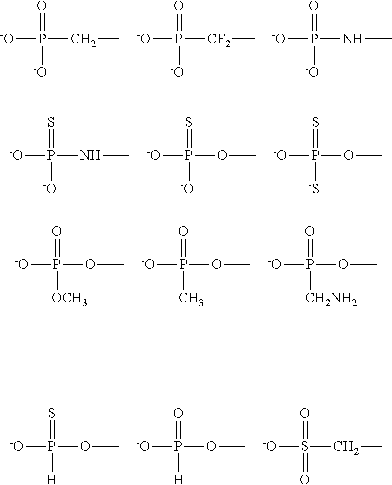

In some embodiments, a 5′-2′ linked nucleotide is a terminal nucleotide and the blunt end or overhang is at the 2′ end. The 5′- and/or 3′-end of one or both strands of the nucleic acid may include a free hydroxyl group. The 5′- and/or 3′-end of a nucleic acid molecule strand may be modified. Examples of 5′ terminal caps include, but are not limited to abasic, deoxy or dideoxy abasic, inverted (deoxy or dideoxy) abasic, glyceryl, dinucleotide, acyclic nucleotide, L-DNA, L-RNA, TNA, and the like. In some embodiments, the 5′ terminal cap (i.e. z″) or 3′ terminal moiety (i.e. Z, Z′ or Z″) comprises a THNB moiety (cf. WO 2014/043291).

The nucleic acid molecules herein may have at least one end of the molecule equipped with an overhang of a nucleotide or non-nucleotide moiety, typically 1-8 such moieties including any integer therebetween. Said nucleotide may be a deoxyribonucleotide, ribonucleotide, natural and non-natural nucleobase or any nucleotide modified in the sugar, base or phosphate group such as disclosed herein. A double stranded nucleic acid molecule may have both 5′- and 3′-overhangs. When two or more the overhangs may be of different lengths. 2-3 unpaired nucleotides are feasible overhangs.

In certain embodiments at least one of the overhang moieties is modified e.g. as a 2′-deoxynucleotide. In other embodiments an overhang includes an alkyl moiety, optionally a propane [(CH₂)₃] moiety (C3) or a derivative thereof, including propanol (C3OH), and phospho derivative of propanediol (“C3-3′Pi”). In some embodiments each of said Z, Z′ and Z″ independently includes two alkyl moieties covalently linked to the 3′ terminus of the antisense or sense strand via a phosphodiester or phosphorothioate linkage and covalently linked to one another via a phosphodiester or phosphorothioate bond, and in some examples is C3Pi-C3Pi or C3Pi-C3OH. A phosphonoacetate bond is an alternative.

Exemplary 3′ terminal non-nucleotide moieties are (B=base):

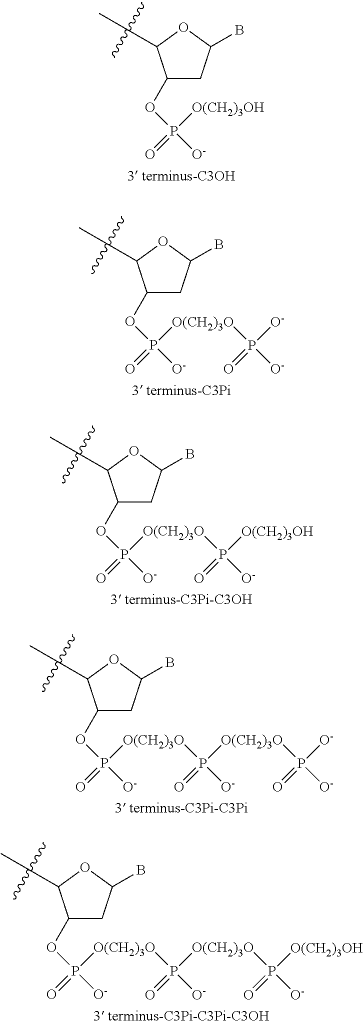

In some embodiments, the A1 moiety is selected from the group consisting of SEQ. ID. NOS: 1-6 (cf. Table 1).

The present oligonucleotide compounds can be synthesized by any method well-known in the art for their synthesis, as described e.g. in Beaucage and Iyer, Tetrahedron 1992; 48:2223-2311. Separate synthesis and post-synthetical joining, e.g. by ligation (Moore et al., 1992, Science 256, 9923), or by hybridization following synthesis and/or deprotection, are also possible.

A commercially available machine (i.a. from Applied Biosystems) can be used for said oligonucleotide synthesis. Overlapping pairs of chemically synthesized fragments can be ligated using methods well known in the art (cf. U.S. Pat. No. 6,121,426). The strands are synthesized separately and then are annealed to each other in the tube.

The present oligonucleotides can also be synthesized via tandem synthesis methodology (cf. US 2004/0019001), wherein both siRNA strands are synthesized as a single continuous oligonucleotide fragment or strand separated by a cleavable linker which is subsequently cleaved to provide separate siRNA fragments or strands that hybridize and permit purification of the siRNA duplex.

Pharmaceutically acceptable salts include mineral or organic acid salts of basic residues such as amines; alkali or organic salts of acidic residues such as carboxylic acids. Typical salts are chlorides, bromides, sulfates, nitrates, phosphates, sulfonates, formates, tartrates, maleates, citrates, benzoates, salicylates, ascorbates, and the like. Sodium salt is preferred.

Furthermore the present disclosure relates to the above disclosed compound having the formula (I), including all embodiments, variations and alternatives thereof discussed and mentioned above, for use as a pharmaceutical.

The present disclosure also relates to a pharmaceutical composition comprising the above disclosed compound having the formula (I), including all embodiments, variations and alternatives thereof discussed and mentioned above, as active ingredient in association with a pharmaceutically acceptable adjuvant, diluent, carrier, solvent, excipient and/or vehicle. Such compounds are often inert, non-toxic materials that do not react with the active ingredient, as is well known to the skilled person.

Furthermore, the present disclosure also relates to the use of the above disclosed compound having the formula (I), including all embodiments, variations and alternatives thereof discussed and mentioned above, for the manufacture of a pharmaceutical composition for treatment of cancer and/or cancer related medical conditions.

As used herein, the term “pharmaceutically acceptable” means that the applicable substance is suitable for use in humans and/or other mammals without undue adverse side effects and commensurate with a reasonable benefit/risk ratio.

The pharmaceutical composition may be adapted for transtympanic, local intraoperative, oral, intravenous, intratumoral, intramuscular, intracerebral, topical, intraperitoneal, nasal, pulmonary, buccal, sublingual or subcutaneous administration or for administration via the respiratory tract e.g. in the form of an aerosol or an air-suspended fine powder. The composition may thus for instance be in the form of microparticles, nanoparticles, liposomes, tablets, capsules, powders, granules, syrups, suspensions, solutions, transdermal patches or suppositories.

The pharmaceutical composition may be adapted for parenteral administration. It may comprise a sterile aqueous preparation of the compounds, which may be isotonic with the blood of the recipient. This aqueous preparation may be formulated according to known methods, using suitable dispersing or wetting agents and suspending agents. The preparation may be a sterile injectable solution or suspension in a diluent or solvent, for example, as a solution in 1,3-butane diol. Water, Ringer's solution, and isotonic sodium chloride solution are exemplary acceptable diluents. Sterile, fixed oils may be employed as a solvent or suspending medium. Bland fixed oils, including synthetic mono- or di-glycerides, and fatty acids, such as oleic acid, may also be used.

The pharmaceutical composition according to the present disclosure may include two or more compounds encompassed by said general formula (I).

The pharmaceutical composition may optionally comprise e.g. at least one further additive selected from a disintegrating agent, binder, lubricant, flavoring agent, preservative, colorant and any mixture thereof. Examples of such and other additives are found in “Handbook of Pharmaceutical Excipients”; Ed. A. H. Kibbe, 3^{rd} Ed., American Pharmaceutical Association, USA and Pharmaceutical Press UK, 2000.

As mentioned above, the present disclosure i.a. relates to the use of the above disclosed compound having the formula (I), including all embodiments, variations and alternatives thereof discussed and mentioned above, for the manufacture of a pharmaceutical composition for treatment of cancer and/or cancer related medical conditions.

The present disclosure also relates to the above disclosed compound having the formula (I), including all embodiments, variations and alternatives thereof discussed and mentioned above, in the treatment of cancer and/or a cancer related medical condition.

The present disclosure further relates to a method for treatment of cancer and/or a cancer related medical condition, wherein said method comprises administering to a patient a therapeutically effective amount of said compound (I).

The patient is preferably a human.

The compound should be administered in a safe and therapeutically effective amount. This refers to the quantity of a component which is sufficient to yield a desired therapeutic response without unacceptable adverse side effects. For example, an amount effective to delay the growth or incidence of a cancer, or to shrink the cancer or prevent metastasis. The specific safe and effective amount or therapeutically effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

The term “cancer” as used herein refers to a proliferative disease or a malignant neoplasm (tumor). Examples include but are not limited to, breast cancer, prostate cancer, ovarian cancer, cervical cancer, skin cancer, pancreatic cancer, colorectal cancer and lung cancer.

The term “cancer related medical condition” as used herein refers to any disease or condition that may be a cause of cancer, a symptom of cancer or a condition wherein the patient has an increased risk of developing cancer.

The typical dosage of compound (I) varies within a wide range and will depend on various factors such as the individual needs of each patient and the route of administration. The dosage administered by infusion is generally within the range of 0.01-200 μg/kg body weight per hour. A physician of ordinary skill in the art will be able to optimize the dosage to the situation at hand.

The compounds or pharmaceutical compositions according to the present disclosure may be administered in a single dose or in multiple doses.

The compounds or pharmaceutical compositions disclosed herein may also be used in combination therapy, when the compound or pharmaceutical composition is administered in combination, either simultaneously or separately, with another compound, such as an additional pharmaceutically active compound, or another therapy, such as chemotherapy or radiotherapy.

The term “RNA interference” or “RNAi” refers to the sequence-specific, post transcriptional silencing or reducing of target gene expression with nucleic acid based molecules, e.g. siNAs including siRNAs and miRNAs acting via interaction with a specific protein complex known as RNA silencing complex (RISC). The target gene may be endogenous or exogenous to the organism; if exogenous, it may be present as integrated into a chromosome, as an episomal DNA (or RNA) in the host cell or outside the host cell. Gene expression is usually either completely or partially inhibited. RNAi is mediated by either double- or single-stranded nucleic acids—“dsNA” and “ssNA”, respectively. In case of dsNA, one of the duplex strands, referred to as the guide strand, is complementary (antisense) to target RNA.

The term “inhibition” of a target gene refers to attenuation, reduction or down-regulation of gene expression or polypeptide activity of a target gene wherein the target gene is selected from a gene transcribed into an mRNA or a single nucleotide polymorphism (“SNP”) or other variants thereof.

The mechanism of antisense (AS) modulation of gene expression depends on the structure of the AS molecule and its target. As an example, AS gapmers contain a DNA region complementary to mRNA to inhibit transcription via RNase H-dependent mechanisms. In another example, modified AS oligonucleotides can regulate alternative splicing if directed towards splice junctions in pre-mRNA.

The term “target RNA” refers to an RNA molecule to which at least one strand of the dsNA or ssNA is homologous or complementary or to which a miRNA possesses homology. Target RNA molecule can be mRNA (messenger RNA) and lncRNA (long non-coding RNA) or lincRNA (large intergenic non-coding RNAs) including but not limited to naturally occurring antisense RNAs (AS RNA) and eRNA (enhancer RNA), as well as pre-miRNA or pro-miRNA. Unprocessed mRNA, ribosomal RNA, and viral RNA sequences may also be targets.

A target RNA is typically modulated by a dsNA or ssNA. Modulation usually refers to post-transcriptional down-regulation (e.g. via RNAi or AS activity) or upregulation (e.g. via anti-miR activity). In some embodiments, ss- or dsNA modulates their target RNA without affecting its levels but rather by modulating their function (e.g. anti-miRs that block miRNA activity). In other embodiments, target RNA is referred to as one the levels of which are affected by ssNA and/or dsNA in the absence of direct sequence homology between the NA and the target. This can happen e.g. in the case of RNAa when activation of target RNA expression is achieved at a transcriptional, rather than at a post-transcriptional, level.

The polynucleotide sequence of the target mRNA sequence, or the target gene having a mRNA sequence refer to the mRNA sequences available in public data bases or any homologous sequences thereof preferably having at least 70% identity, or 80% identity, or 90% or 95% identity to any one of mRNA. Therefore, polynucleotide sequences derived from mRNA sequences which have undergone mutations, alterations or modifications are encompassed by the present disclosure.

In some embodiments the target gene is a mammalian gene, and in variants of these embodiments, the target gene is a human gene.

The compounds herein inhibit gene function (examined by e.g. enzymatic assay), including inhibition of protein (examined by e.g. Western blotting) and inhibition of mRNA expression (examined by e.g. quantitative RT-PCR).

### EXAMPLES

The following is to be construed as illustrative and not as a limitation of the subject-matter specified in the claims.

Standard molecular biology protocols used are known in the art.

In the examples, eight different oligonucleotide moieties are used, SEQ. ID. NOS: 1-10, of these SEQ. ID. NOS: 7-8 are used as controls. These are shown inter alia in the Table 1 below, where all sequences have a length of 19 base pairs.

Furthermore, in the examples, different moieties are conjugated to the nucleotide moiety. In addition to the Q10 moiety (cf. FIG. 1) these are cyanine dye and sphingolipid-spermine phosphoramidite, as shown below:

### Cyanine Dye (Cy3 Phosphoramidite):

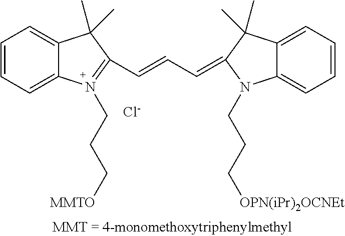

### Sphingolipid-Spermine Phosphoramidite:

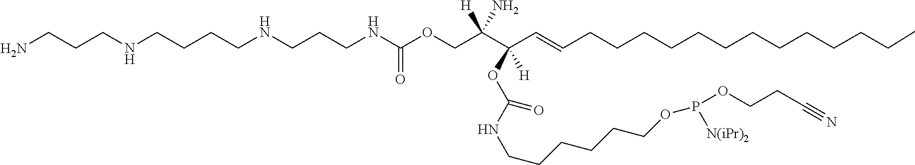

TABLE 1

| Duplex Name | Sense Description | Antisense Description |
|---|---|---|
| RAC1_28_S2045 | 1, 3, 5, 10, 13, 16, 18-2′-OMe- | 1, 6, 9, 11, 13, 15, 17, 19-2′- |
| | 3′-Pi; 0-cap-SL-Spermine-pi; | OMe-3′-Pi; Phosphate |
| | Phosphate | |
| RAC1_28_S2281 | 1, 3, 5, 10, 13, 16, 18-2′-OMe- | 1, 6, 9, 11, 13, 15, 17, 19-2′- |
| | 3′-Pi; 0-cap-SL-Spermine-pi; | OMe-3′-Pi; 20-Cy3 |
| | Phosphate | |
| RAC1_28_S2503 | 1, 3, 5, 10, 13, 16, 18-2′-OMe- | 1, 6, 9, 11, 13, 15, 17, 19-2′- |
| | 3′-Pi; 0-cap-Q10-C6NH2 | OMe-3′-Pi; Phosphate |
| RAC1_28_S2504 | 1, 3, 5, 10, 13, 16, 18-2′-OMe- | 1, 6, 9, 11, 13, 15, 17, 19-2′- |
| | 3′-Pi; 0-cap-Q10-C6NH2 | OMe-3′-Pi; 20-Cy3 |
| RAC1_28_S2553 | 1, 3, 5, 10, 13, 16, 18-2′-OMe- | 1, 6, 9, 11, 13, 15, 17, 19-2′- |
| | 3′-Pi; 0-cap-Q10-C6NH2 | OMe-3′-Pi; 0-5′ |
| | | phophate; Phosphate |
| RAC1_28_S1908 | 1, 3, 5, 10, 13, 16, 18-2′-OMe- | 1, 6, 9, 11, 13, 15, 17, 19-2′- |
| | 3′-Pi; Phosphate | OMe-3′-Pi; Phosphate |
| RAC1_28_S2132 | 1, 3, 5, 10, 13, 16, 18-2′-OMe- | 1, 6, 9, 11, 13, 15, 17, 19-2′- |
| | 3′-Pi; Phosphate | OMe-3′-Pi; 20-Cy3 |
| Strand name | Description | |
| RAC1_28_F_1539 | 1, 3, 5, 10, 13, 16, 18-2′-OMe- | |
| | 3′-Pi; 20-tail-C6NH2-Q10 | |
| Duplex Name | Sense 53 | Antisense 53 |
| RAC1_28_S204 | zSLSp; mC; rG; mU; rG; mC; rA; | mU; rA; rG; rG; rA; mU; rA; rC; mC; |
| 5 | rA; rA; rG; mU; rG; rG; mU; rA; | rA; mC; rU; mU; rU; mG; rC; mA; rC; |
| | rU; mC; rC; mU; rA | mG |
| | (SEQ ID NO: 1) | (SEQ ID NO: 2) |
| RAC1_28_5228 | zSLSp; mC; rG; mU; rG; mC; rA; | mU; rA; rG; rG; rA; mU; rA; rC; mC; |
| 1 | rA; rA; rG; mU; rG; rG; mU; | rA; mC; rU; mU; rU; mG; rC; mA; rC; |
| | rA; rU; mC; rC; mU; rA | mG; zcy3$ |
| | (SEQ ID NO: 3) | (SEQ ID NO: 4) |
| RAC1_28_5250 | zCoQ10; zc6NH2; mC; rG; mU; rG; | mU; rA; rG; rG; rA; mU; rA; rC; mC; |
| 3 | mC; rA; rA; rA; rG; mU; rG; rG; | rA; mC; rU; mU; rU; mG; rC; mA; rC; |
| | mU; rA; rU; mC; rC; mU; rA$ | mG |
| | (SEQ ID NO: 5) | (SEQ ID NO: 6) |
| RAC1_28_5250 | zCoQ10; zc6NH2; mC; rG; mU; rG; | mU; rA; rG; rG; rA; mU; rA; rC; mC; |
| 4 | mC; rA; rA; rA; rG; mU; rG; rG; | rA; mC; rU; mU; rU; mG; rC; mA; rC; |
| | mU; rA; rU; mC; rC; mU; rA$ | mG; zcy3$ |
| | (SEQ ID NO: 7) | (SEQ ID NO: 8) |
| RAC1_28_5255 | zCoQ10; zc6NH2; mC; rG; mU; rG; | 5′p; mU; rA; rG; rG; rA; mU; rA; rC; |
| 3 | mC; rA; rA; rA; rG; mU; rG; rG; | mC; rA; mC; rU; mU; rU; mG; rC; mA; |
| | mU; rA; rU; mC; rC; mU; rA$ | rC; mG |
| | (SEQ ID NO: 9) | (SEQ ID NO: 10) |
| RAC1_28_S 190 | mC; rG; mU; rG; mC; rA; rA rA; | mU; rA; rG; rG; rA; mU; rA; rC; mC; |
| 8 | rG; mU; rG; rG; mU; rA; rU; mC; | rA; mC; rU; mU; rU; mG; rC; mA; rC; |
| | rC; mU; rA | mG |
| | (SEQ ID NO: 11) | (SEQ ID NO: 12) |
| RAC1_28_5213 | mC; rG; mU; rG; mC; rA; rA; rA; | mU; rA; rG; rG; rA; mU; rA; rC; mC; |
| 2 | rG; mU; rG; rG; mU; rA; rU; mC; | rA; mC; rU; mU; rU; mG; rC; mA; rC; |
| | rC; mU; rA | mG; zcy3$ |
| | (SEQ ID NO: 13) | (SEQ ID NO: 14) |
| Strand name | 5-+223 | |
| RAC128_F_15 | mC; rG; mU; rG; mC; rA; rA; rA; | |
| 39 | rG; mU; rG; rG; mU; rA; rU; mC; | |
| | rC; mU; rA; zc6NH2; zCoQ10$ | |
| | (SEQ ID NO: 15) | |

TABLE 2
Legends for compounds of Table 1

| Sense/ | | |
|---|---|---|
| Antisense | Modification | |
| Description | Code | Description |
| RNA-3′-Pi | r | ribonucleotide-3′-phosphate |
| | rA | riboadenosine-3′-phosphate; 3′-adenylic acid |
| | rC | ribocytidine-3′-phosphate; 3′-cytidylic acid |
| | rG | riboguanosine-3′-phosphate; 3′-guanylic acid |
| | rU | ribouridine-3′-phosphate; 3′-uridylic acid |
| 2′-OMe-3′-Pi | m | 2′-O-methyl-ribonucleotide-3′-phosphate |
| | mA | 2′-O-methyladenosine-3′-phosphate; 2′-O- |
| | | methyl-3′-adenylic acid |
| | mC | 2′-O-methylcytidine-3′-phosphate; 2′-O- |
| | | methyl-3′-cytidylic acid |
| | mG | 2′-O-methylguanosine-3′-phosphate; 2′-O- |
| | | methyl-3′-guanylic acid |
| | mU | 2′-O-methyluridine-3′-phosphate; 2′-O- |
| | | methyl-3′-uridylic acid |
| -Pi | | Component of the modification (used to- |
| | | gether with above Phosphoramidites) |
| Phosphate | | Phosphate at the 3′ end of the sequence |
| — | $ | No Phosphate at the 3′ end of the sequence |
| cap or tail | z | The general legend for the description of any |
| | | overhangs at the 3′ (=cap) or 5′ (=tail) |
| | | of the sequence |
| cap or tail - | zcy3 | Cyanine Dye at the 3′-end or 5′-end of the |
| Cy3 | | sequence |
| cap-SL- | zSLSp | Sphingo-Lipid-Spermine-3′-phosphate at the |
| Spermine-pi | | 3′-end of the sequence |
| cap or tail - | zCoQ10 | Coenzyme Q10 at the 3′-end or 5′-end of the |
| coenzyme | | sequence (also known as ubiquinone) |
| Q10 | | |

### 1. Experimental (Synthesis)

Unless otherwise provided, reactions were performed at room temperature. The purity of the synthesized compounds was usually determined by chromatography (TLC, HPLC) and NMR. Mass spectrometry analysis was also utilized to confirm product identity.

### Example 1.1

### Synthesis of Q10 NHS Activated Ester

a. 6-amino-1-hexanol (10 g), compound 1, was dissolved in dry dichloromethane, DCM, under argon with stirring. Ethyl trifluoroacetate (EtOTf; 11 ml) was added, and the solution was further stirred until the reaction was completed. Solvent was evaporated under reduced pressure to obtain crude product. Crude material was purified using column chromatography (silica gel, DCM/MeOH as eluent) to provide compound 2.

b. Compound 2 (15 g) was dissolved in dry pyridine under argon with stirring. p-toluenesulphonyl chloride (TsCl; 1 eq.) was added, and the reaction was stirred until completion. The reaction mixture was worked-up and concentrated to dryness. Crude material was purified using column chromatography (silica gel, DCM/MeOH as eluent) to provide compound 3.

c. Compound 3 (24 g) was dissolved in acetone, under argon with stirring. Potassium iodide was added and the reaction mixture was heated and stirred until completion. Reaction mixture was worked-up and concentrated to dryness. Crude material was purified using column chromatography (silica gel, DCM/MeOH as eluent) to provide compound 4.

d. Compound 5 Q10 (5 g), ubidecarenone, was dissolved in diethylether (150 ml) with stirring and an aqueous solution of Na₂S₂O₄ (5 g) was added. The mixture was stirred until reaction completion. Reaction mixture was worked-up and concentrated to dryness. Crude material was purified using column chromatography (silica gel, hexane/ethyl acetate as eluent) to provide compound 6.

e. Compound 6 was dissolved in tetrahydrofuran, THF. Sodium hydride (0.93 g, 60% in mineral oil) was added and the mixture was stirred for a few min. A solution of compound 4 (7.47 g) was then added, and reaction was stirred until completion. Crude material was purified using column chromatography (silica gel, petroleum ether/ethyl acetate as eluent) to provide compound 7.

f. Compound 7 (1.25 g) was dissolved in MeOH/DCM. A solution of aqueous NaOH was added and the reaction mixture was stirred until completion. Reaction mixture was worked up and concentrated under reduced pressure. Crude material was purified using column chromatography (silica gel, hexane/ethyl acetate as eluent) to provide compound 8.

g. Compound 8 (1.05 g) was dissolved in pyridine, and imidazole and tert-butyldimethylsilyl chloride (TBDMSCl; 3 to 5 eq.) were added. Reaction mixture was stirred until completion, worked-up and concentrated under reduced pressure. Crude material was purified using column chromatography (silica gel, MeOH/DCM as eluent) to provide compound 9.

h. Compound 9 (1.46 g) was dissolved in DCM and dimethylaminopyridine (DMAP; 165 mg) was added followed by succinic anhydride (1 eq.), and the reaction mixture was stirred until completion, then worked-up and concentrated under reduced pressure. Crude material was purified using column chromatography (silica gel, MeOH/DCM as eluent) to provide compound 10.

i. Compound 10 (1.45 g) was dissolved in DCM, and dicyclohexyl-carbodiimide (DCC; 1.2 eq.) and N-hydroxysuccinimide (NHS; 1.2 eq.) were added. The reaction mixture was worked-up and concentrated under reduced pressure. The below product compound 11 (cf. also FIG. 1) was purified using column chromatography.

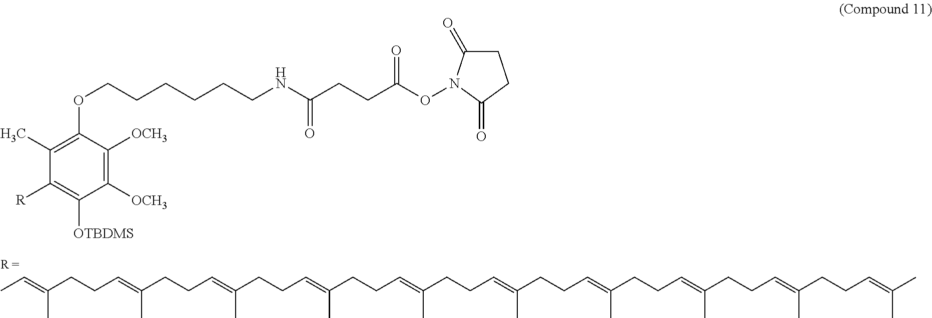

The above steps a.-i. to prepare the compound 11, with side chain attached to Q10, are illustrated in the below general reaction scheme, with ‘R’ as above:

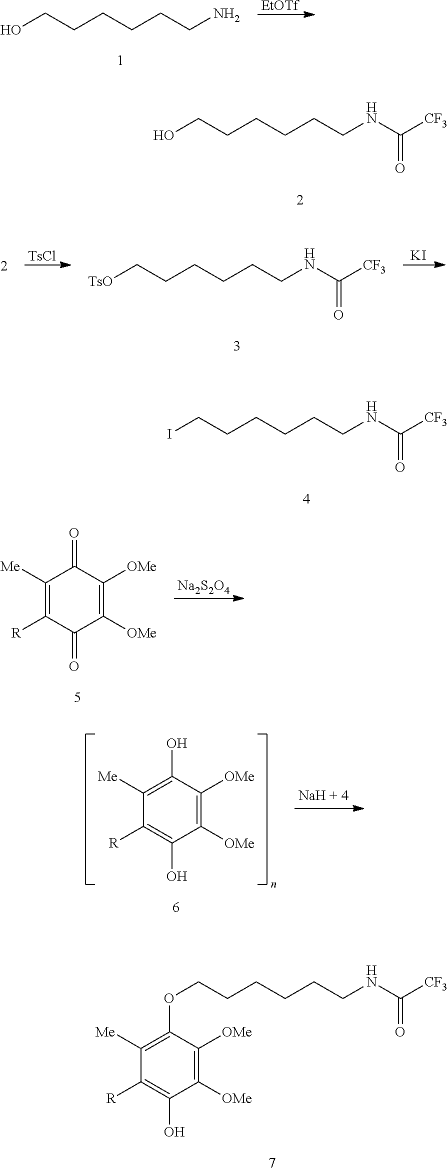

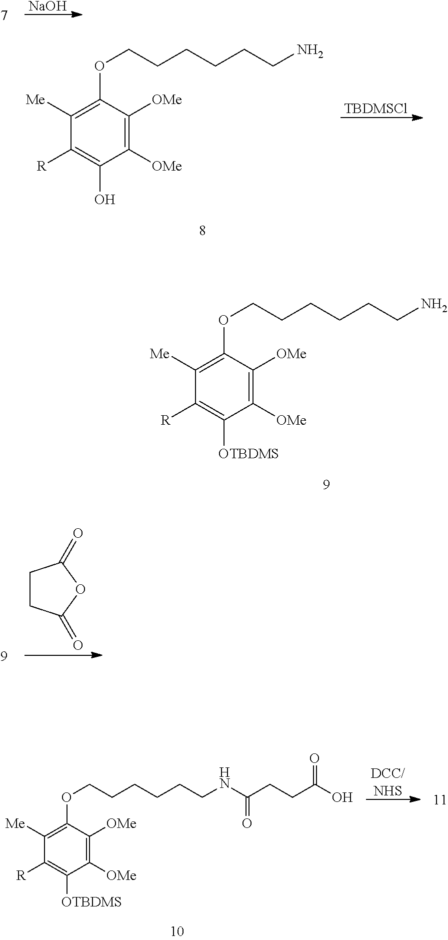

The Q10 NHS activated ester 11 was stored at −20° C. before use in the examples below.

### Example 1.2

### Synthesis of RNA Q10-Conjugate

The synthetic approach to access the desired Q10 RNA conjugate is based on an amide bond formation of the N-hydroxysuccinimide (NHS) activated ester of Q10 and the RNA equipped with a hexylamine linker immobilized on a solid support.

Table 3 contains the RNA sequence information.

TABLE 3
In this table, lower case letters a, c, g, u, are
2′-O-Methyl nucleotides; Upper case letters A, C,
G, U represent RNA nucleotides. (NH2C6) denotes
the aminohexyl linker.

| | Molecular |
|---|---|
| Sequence 5′-3′ | weight (D) |
| (NH₂C6)cGuGcAAAGuGGuAUcCuA | 6339.9 |
| (SEQ ID NO: 16) | |
| (Q10)(NH₂C6)cGuGcAAAGuGGuAUcCuA | 7386.6 |
| (SEQ ID NO: 17) | |

The RNA sequence was synthesized according to the phosphoramidite technology on solid phase. The synthesis was performed on an Expedite 8909 synthesizer (Applied Biosystems) with controlled pore glass (CPG, 520 Å, with a loading of 35 μmol/g, obtained from Prime Synthesis, Aston, Pa., USA) serving as the solid support. Ancillary synthesis reagents, RNA as well as 2′-O-Methyl RNA phosphoramidites were obtained from SAFC Proligo (Hamburg, Germany). Specifically, 5′-O-(4,4′-dimethoxytrityl)-3′-O-(2-cyanoethyl-N,N-diisopropyl)phosphoramidite monomers of uridine (U), 4-N-acetylcytidine (CAc), 6-Nbenzoyladenosine (Abz) and 2-N-isobutyrlguanosine (GiBu) with 2′-O-t-butyldimethylsilyl protection were used to build the oligoribonucleotide sequence. 2′-O-Methyl modifications were introduced employing the corresponding phosphoramidites carrying the same nucleobase protecting groups as the regular RNA building blocks. All phosphoramidites were dissolved in anhydrous acetonitrile (100 mM) and molecular sieves (3 Å) were added. Coupling times were 5 minutes with 5-Ethyl thiotetrazole (ETT, 500 mM in acetonitrile) as activator solution. In order to introduce the aminohexyl linker at the 5′-end of the oligomer the 6-(4-Monomethoxytritylamino)hexyl-(2-cyanoethyl)-(N,N-diisopropyl)-phosphoramidite from SAFC Proligo was employed. Coupling time for this modification was 12 min and no capping was employed for this coupling. The monomethoxytrityl (MMT) group on the primary amine was removed by pumping the deblock solution (3% trichloroacetic acid in dichloromethane) through the synthesis column as long as no more yellow color of the MMT cation was visible. Finally, the solid support was washed thoroughly with anhydrous acetonitrile and dried in stream of argon.

Subsequent to the completion of the solid phase synthesis 70 mg solid support was transferred to a 2 mL polypropylene screw cap vial (Sorenson BioScience, Salt Lake City, Utah, USA) to prepare the Q10-conjugate.

Compound 11 (100 mg) was thawed and dissolved in 900 μL dry dichloromethane (Sigma Aldrich, Traufkirchen, Germany). 450 μL of this solution was added to the CPG followed by 50 μL N,N-diisopropylethylamine (DIPEA, Sigma Aldrich). The suspension was shaken on an Eppendorf Thermomixer (Eppendorf, Hamburg, Germany) at 25° C. over night.

The next day the suspension was diluted by the addition of 1 mL dichloromethane. The vial was centrifuged at 13200 rpm in a centrifuge (centrifuge 5415R Eppendorf, Hamburg, Germany) for 5 minutes. The supernatant was removed and discarded. The CPG was washed by the addition of 1 mL DCM. Centrifugation was repeated and the supernatant again discarded. Next, the oligonucleotides was cleaved from the solid support and deprotected using a 1:1 (v/v) mixture of methylamine in water (40%, Sigma Aldrich) and aqueous ammonia (30%, Aldrich). For this purpose, the solid support was treated for 2.5 hour at 25° C. with the deprotection cocktail. Subsequently, the vial was centrifuged in an Eppendorf centrifuge at 13200 rpm and the supernatant was transferred to a new vial. The CPG was washed with dimethyl sulfoxide (DMSO) and the solution was combined with the RNA solution in the new vial. Tert-butyldimethylsilyl cleavage was accomplished by the addition of triethylamine trihydrofluoride (Merck, Darmstadt, Germany) and incubation for 90 minutes at 45° C. The reaction was quenched by the addition of a 1-Methyl-2-pyrrolidinone (NMP, Sigma Aldrich)/ethanol/ethoxytrimethylsilane (Merck) mixture (1/4/2 v/v) and the precipitated oligonucleotide was isolated by centrifugation.

The pellet was dissolved in 100 mM triethylammonium acetate (TEAAc, Biosolve, Valkenswaard, The Netherlands) and purified using a C4-RP HPLC column (5 μm, 100×10 mm, YMC, Dinslaken, Germany). An AKTA Purifier HPLC system with fraction collector (GE Healthcare, Freiburg, Germany) was employed. The crude reaction mixture was purified by gradient elution using 100 mM TEAAc as Eluent A and 100 mM TEAAc in 95% acetonitrile as Eluent B. A gradient from 5% eluent B to 100% eluent B in 25 minutes was used. Flow rate was 4 mL/min (approximately 305 cm/h) and fraction size was 1 mL. The elution was monitored at 260 and 280 nm. The desired conjugate elutes at about 80% eluent B.

Fractions containing the desired product were combined and precipitated at −20° C. overnight using 3M sodium acetate (pH 5.2) and ethanol. The pellet was dissolved in water and the concentration was determined in an Eppendorf photometer at 260 nm. 44 ODs(260) have been isolated. Purity (see Table 4) was analyzed using a Dionex Ultimate 3000 HPLC system (Dionex, Idstein, Germany) equipped with an analytical C4 Acquity BEH 300 column (1.7 μm, 2.1×100 mm Waters, Eschborn, Germany). Identity was confirmed by electrospray mass spectrometry (ESI-MS) using a LCQ Deca XP Plus Instrument from Thermo Fisher.

TABLE 4
Analytical data for the Q10 conjugate

| | Purity | Mol weight | Mol weight |
|---|---|---|---|
| Sequence 5′-3′ | (% RP HPLC) | (measured) | (calc) |
| S(Q10)(NH₂C6)cGuGcAAAGuGGuAUcCuA | 93.0 | 7386.6 | 7387.5 |
| (SEQ. ID. NO: 17) | | | |

### Example 1.3

### Synthesis of dsRNA Compounds

Annealing was carried out using standard methodology by treatment of two strands at 1:1 molar ratio at 250 μM concentration in a PBS buffer at 85° C. for 10 minutes, followed by allowing the solution to cool to ambient room temperature for about 2 hours.

### 2. Experimental (Biological Testing)

### Example 2.1

### In Vitro Knockdown Activity Study

In vitro knockdown activity of Q10-conjugated siRNA RAC1_28_S2503 and non-conjugated siRNA RAC1_28_S1908 was analyzed. Target knockdown activity was studied using the psiCHECK™ expression system (Promega) that enables the evaluation of the intrinsic potency of inhibitory oligonucleotides by monitoring the changes in the activity of Luciferase reporter gene carrying the target sites for inhibitory oligonucleotide action in its 3′ untranslated region (3′-UTR). The activity of a siRNA toward this target sequence results either in cleavage and subsequent degradation of the fused mRNA (the most likely scenario) or in translation inhibition of the encoded protein. In addition, the psiCHECK™-2 vector contains a second reporter gene, Firefly luciferase, transcribed from a different promoter and non-affected by the inhibitory oligonucleotide under study. This allows for normalization of Renilla luciferase expression across different transfections. psiCHECK™-2-based construct was prepared for the evaluation of the on-target activity of the guide strands (GS) of RAC1 siRNAs. In the construct, one copy of the full target sequence of the test molecules GS was cloned into the multiple cloning site located in the 3′-UTR of the Renilla luciferase, downstream to the stop codon. The psiCHECH™-2 plasmid was transfected into human HeLa cells. The transfected HeLa cells were then seeded into the wells of a 96-well plate and incubated at 37° C. with the siRNA in duplicates with formulated with Lipofectamine 2000 (protocol according to manual) transfection reagent. Concentrations of the RAC1 siRNAs tested were 0.0061, 0.098, 0.39, 1.56, 6.25 and 100 nM. Control cells were not exposed to any siRNA. 48 hours following siRNA addition, the cells were harvested for protein extraction. Renilla and FireFly Luciferase activities were measured in individual cell protein extracts using Dual-Luciferase® Assay kit according to the manufacturer procedure. Renilla Luciferase activity values were normalized by Firefly Luciferase activity values obtained from the same samples. siRNA activity was expressed as percentage of residual normalized Renilla Luciferase activity in a test sample from the normalized Renilla Luciferase activity in negative control cells.

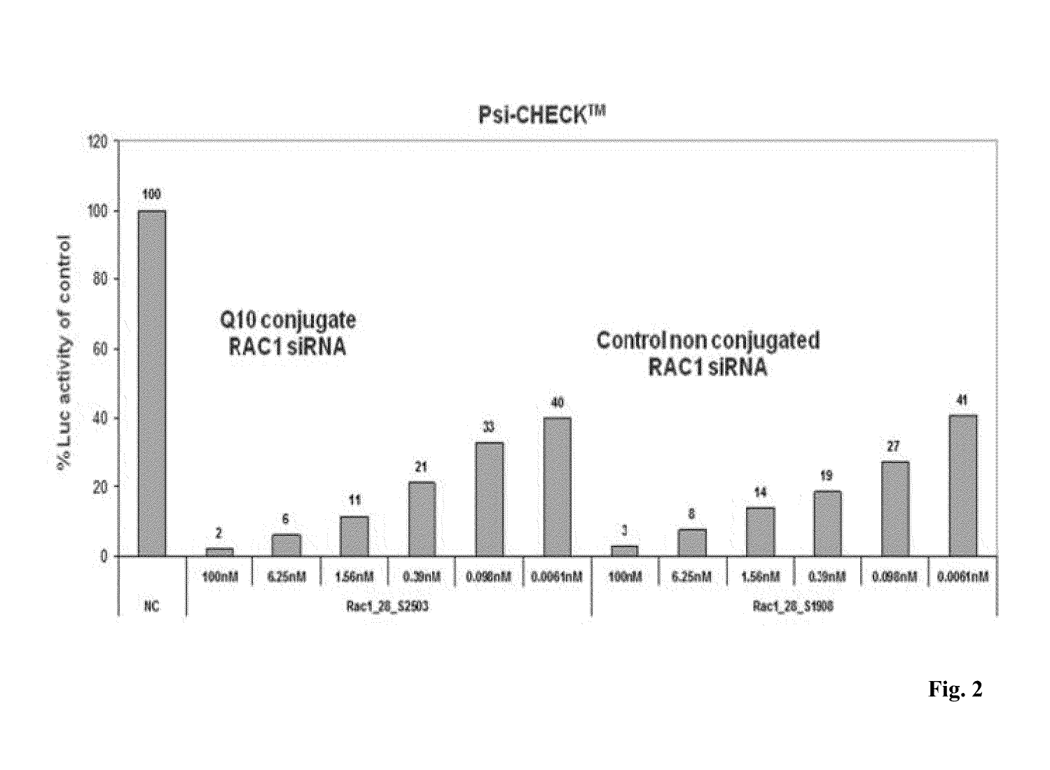

The study was done three times and averaged representative results are shown in FIG. 2.

As shown in FIG. 2, dose-dependent knockdown of Renilla Luciferase activity was demonstrated for Q10-conjugated siRNA. The activity was similar to the non-conjugated siRNA.

### Example 2.2

### Stability Study

The stability of RAC1_28_S2503 against degradation by nucleases was analyzed by incubation for 24 hours at 37° C. in mouse plasma, rat plasma and LLC1 cell extract. At time points between 0 and 24 hours after incubation, 1 ng aliquots were transferred to TBE-loading buffer, snap frozen in liquid nitrogen and stored at −20° C. until use. The aliquots were thawed on ice and analyzed by non-denaturing polyacrylamide gel electrophoresis.

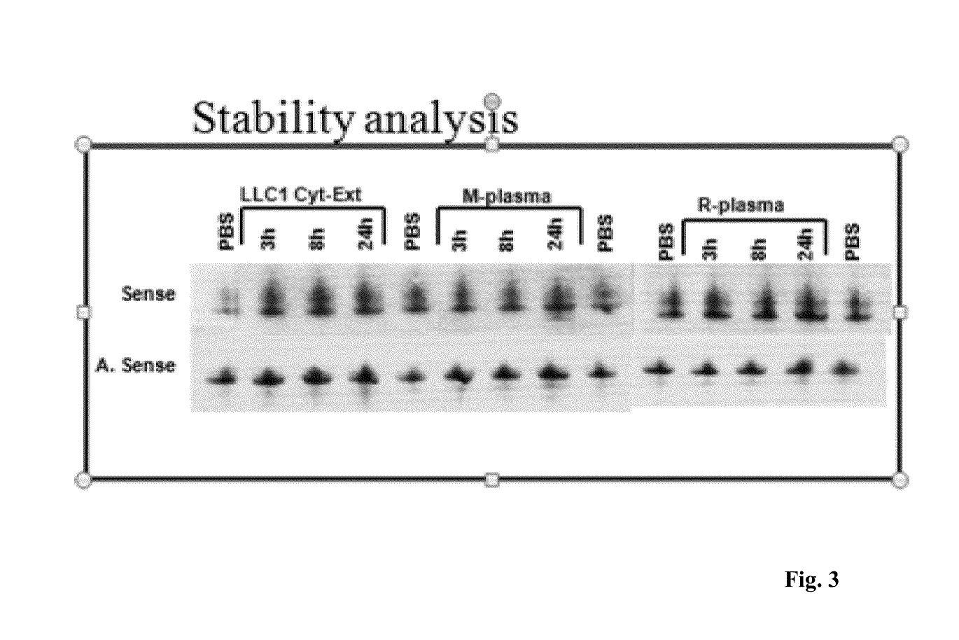

Based on the gel migration patterns (cf. FIG. 3) RAC1_28_S2503 was stable for at least 24 hours at 37° C. in plasma and cell extract.

### Example 2.3

### Comparative Pharmacokinetic Study

The Pharmacokinetics (pK) of Q10 conjugated RAC1_28_S2503 in plasma was compared to the non-conjugated RAC1_28_S1908 and to Sphingolipid Spermine-conjugated RAC1_28_S2045 following i.v. administration of 4 mg/kg siRNA to mice. At 10 min, 2 h, 4 h, 8 h and 24 h after the siRNA administration, blood samples (around 50 μl of total volume from tail) were collected into EDTA collecting tubes. Collected blood samples obtained from all animals were processed for plasma separation by centrifugation (2500 g, for 15 minutes at room temperature). The siRNA was extracted from the plasma using Triton X-100 extraction. For determining the RAC1 siRNA levels in the samples cDNA was prepared using the Stem loop method for siRNA detection. qPCR was carried out using QBI SOP 60-40-02. In a slight variation to the protocol the SYBR fast ABI prism Ready mix kit (KAPA cat no. KKKK4605) was used with an elongation/extension time of 30 seconds. 0.4 μl of each primer and 6.2 μl of water was used per sample in the reaction mix.

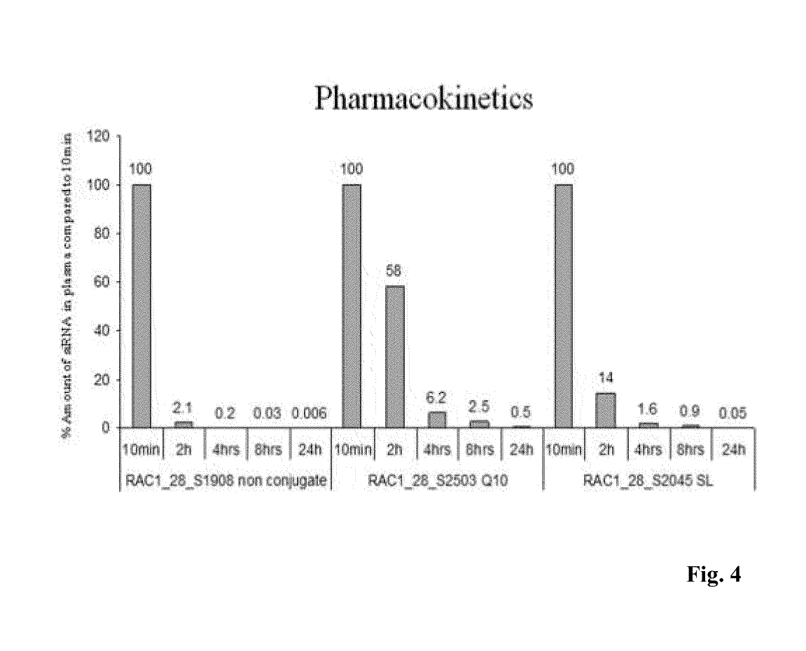

The results are presented in FIG. 4. As can be seen in FIG. 4 the residual level of the Q10-conjugated siRNA was at least 25-fold higher compared to the non-conjugated siRNA and about 3-10 fold higher compared to the Sphingolipid conjugated siRNA, respectively. These results were very surprising to the inventors.

### Example 2.4

### Cell Penetration of Q10-Conjugated siRNA Cy3 Labeled siRNA, Microscopy

The purpose of this study was to determine penetration of Q10-conjugated Cy3 siRNA RAC1_28_S2504 and non-conjugated Cy3 siRNA RAC1_28_S2132 to cells. In the study HFL1 cells were incubated with 100 nM Q10-conjugated or with non-conjugated Cy3 labeled siRNA for 24 h, 48 h and 72 h. siRNA treatments were followed by immunofluorescently staining (IF) with either early endosome marker—(EEA1), Late endosome marker (M6P) or mitochondrial marker (MTC). Cells were analyzed in order to define co-localization of both components along the tested time points.

Stained cells were analyzed under ApoTome optical sectioning in the fluorescent microscope.

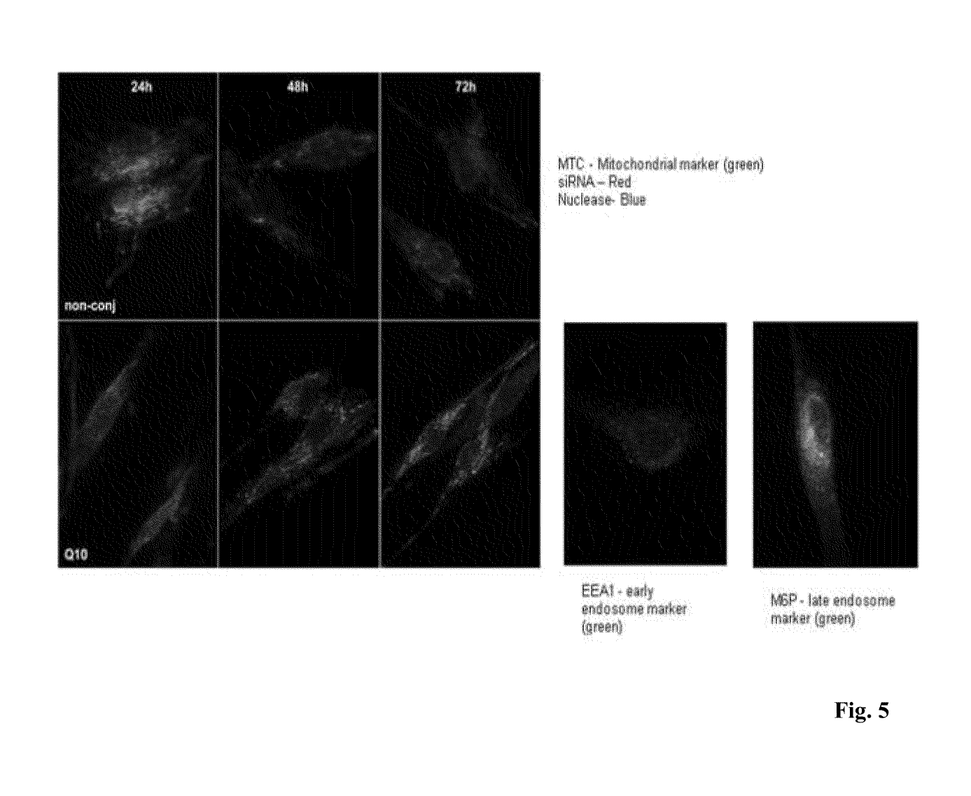

The results of the fluorescence analysis revealed that Q10-conjugated siRNA penetrated into the cells and remained up to 72 hours while the non-conjugated siRNA was not detected at that time point. Immunofluorescence with specific mitochondrial or endosomal markers (early and late endosome) demonstrated that Q10 conjugated siRNA is not co-localized with tested organelles, suggesting that the conjugated siRNA is distributed in the cytoplasm. These results are shown in FIG. 5.

### Example 2.5

### Cell Internalization Kinetics by FACS

In the present study the internalization kinetics of the Q10-conjugated siRNA RAC1_28_S2504 was analyzed. HeLa cells were grown in DMEM, supplemented with 10% fetal bovine serum 4 mM L-Glutamine at 37° C. with 5% CO₂.

The cells were seeded in 6-well tissue culture plates a day before treatment. The staining procedure included incubation of cells with 100 nM of either RAC1_28_S2504 or non-conjugated control RAC1_28_S2132 for 0.5 h, 2 h, and 6 h, respectively. Subsequently, cell media was removed, and the cells were washed in 1 ml PBS and centrifuged at 1400 rpm for 5 min. Cells were then resuspended in PBS and Cy3 siRNA detection in HeLa cells was observed by FACS. The cells were gated using forward (FSC-H)-versus side-scatter (SSC-H) to exclude debris and dead cells and Cy3 intensity was measured by FACScalibur using a FL-2 filter.

The quenching of external fluorescence, which distinguishes internalized from surface-adherent particles, can be accomplished with the use of vital dyes such as trypan blue (TB), which are incapable of penetrating intact cell membranes. In order to distinguish between siRNA molecules that are internalized and are inside the cells from siRNA that is bound to the cells membrane, TB quenching protocol was used. The cells were incubated with 50 μl of 0.4% Trypan Blue for 10 min at RT, to allow quenching of extracellular Cy3 signal. Following this treatment only the Cy3 signal from siRNA that is in the cell can be observed.

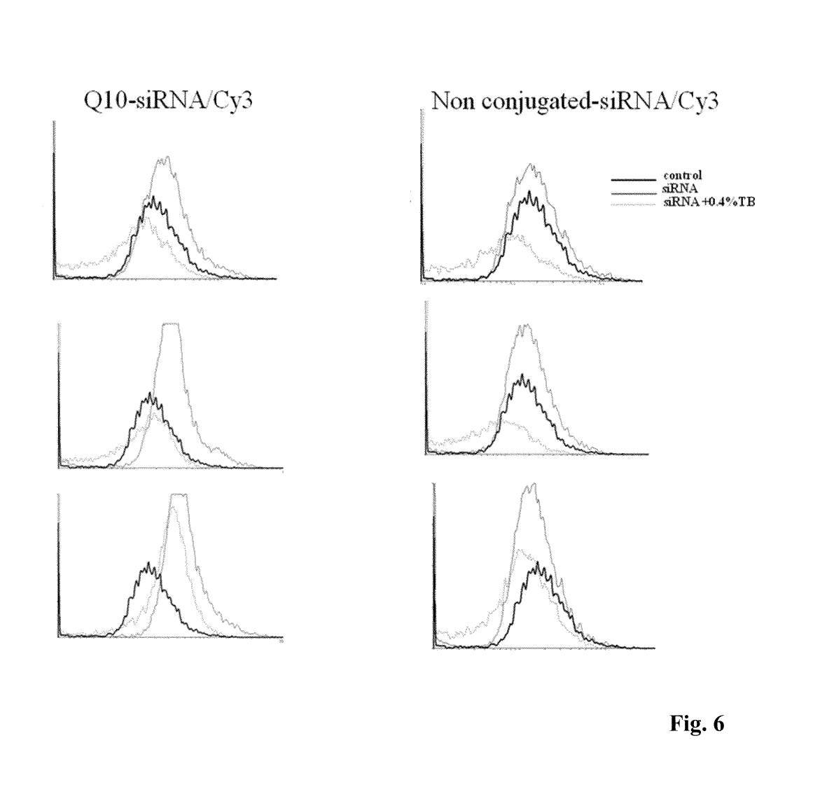

As can be seen in FIG. 6, a shift in cell signal can be observed in cells treated with the conjugated siRNA already after 2 h (middle panels) suggesting its binding to the cells. This shift is increased reaching full staining of most of the cells after 6 h. This shift is hardly observed in the histogram for the cells treated with the non-conjugated siRNA. The FACS analysis presented in FIG. 6 (bottom panels) of cells treated with TB also shows signal shift of cells treated with conjugated siRNA at 6 h of incubation, suggesting that RAC1_28_S2504 was internalized and is found inside the cells. This cell signal shift is not seen in the analysis of the cells treated with RAC1_28_S2132, thereby suggesting inability of non-conjugated siRNA to penetrate the cells.

We claim:

1. A compound having formula (I):
Q-L-A1 (I)
wherein:
Q is a Q10 moiety;
L, which is optionally included, is a linker selected from the group consisting of polyesters, polyethers, polyamines, polyamides, peptides, carbohydrates, lipids, C₃₋₁₂ alkyl straight chain based linkers, polyethylene glycols and other polymeric compounds; and
A1 is a nucleotide moiety;
and pharmaceutically acceptable salts thereof.

2. The compound according to claim 1, wherein Q is ubiquinone.

3. The compound according to claim 1, wherein the linker, when present, is selected from the group consisting of polylactate, triethyloxy-glycol phosphoramidite, ethane-diol-phosphoramidite, hexane-diol phosphoramidite, nonane-diol phosphoramidite, propane-diol phosphoramidite, hexa-ethyloxy-glycol-phosphoramidite and abasic phosphoramidite.

4. The compound according to claim 1, wherein the linker, when present, is selected from the group consisting of polyethylene glycols.

5. The compound according to claim 1, wherein the linker, when present, is derived from a N-hydroxysuccinimide (NHS) ester.

6. The compound according to claim 1, wherein A1 is an oligonucleotide selected from the group consisting of siRNA, ASO, miRNA, antimir, ribozyme, mRNA, and aptamers.

7. The compound according to claim 1, wherein A1 is a double stranded oligonucleotide.

8. The compound according to claim 1, wherein A1 is selected from the group consisting of siRNA, miRNA, miRNA mimetic and modified versions thereof.

9. The compound according to claim 1, wherein A1 is a siRNA or a modified siRNA.

10. The compound according to claim 1, wherein A1 is:
5′ Z″-N1-(N)x-Z 3′ (antisense strand)
3′ Z′-N2-(N′)y - z″ 5′ (sense strand)
wherein each of N1, N2, N and N′ independently is an unmodified nucleotide, a modified nucleotide, a nucleotide analogue or an unconventional moiety;
wherein each of (N)ₓ and (N′)_{y} is an oligonucleotide in which each consecutive N and N′ is joined to the adjacent N or N′ by a covalent bond;
wherein each of x and y is, independently, an integer from 14 to 48;
wherein at least a portion of the sequence of N2-(N′)_{y} is complementary to at least a portion of the sequence of N1-(N)ₓ and at least a portion of the sequence of (N)ₓ is complementary to a consecutive sequence in a target RNA;
wherein N2 is covalently bound to (N′)_{y};
wherein N1 is covalently bound to (N)ₓ and is matched or mismatched to the target mRNA;
wherein z″, optionally present, is a covalently attached capping moiety or a covalent bond to the Q moiety or to the linker L; and
wherein each of Z, Z′, and Z″ comprises 1-2 consecutive non-nucleotide moieties;
wherein each of Z, Z′, and Z″, optionally present, is independently as covalently attached 1-5 consecutive nucleotides, 1-5 consecutive nucleotide analogues or 1-5 consecutive non-nucleotide moieties, or a covalent bond to the Q moiety or to the linker L, or a combination thereof.

11. The compound according to claim 10, wherein the covalent bond joining each consecutive N and/or N′ is independently selected from the group consisting of a phosphodiester bond, a phosphorothioate bond and a modified internucleotide linkage.

12. The compound according to claim 10, wherein x and y are of the same length.

13. The compound according to claim 10, wherein both x and y are 18-25.

14. The compound according to claim 10, wherein both x and y are 18.

15. The compound according to claim 10, wherein the sequence of (N′)_{y} is fully complementary to the sequence of (N)ₓ, and the sequence of (N)ₓ is fully complementary to a target RNA.

16. The compound according to claim 10, wherein x and y are of different lengths, and wherein x is 18-25 and y is 15-17.

17. A pharmaceutical composition comprising a compound according to claim 1; and a pharmaceutically acceptable adjuvant, diluent or carrier.

18. A method for treatment of cancer and/or a cancer related medical condition, comprising administering to a patient in need of said treatment a therapeutically effective amount of a compound according to claim 1.
